(19) United States
(12) Patent Application Publication
(10) Pub. No.: US 2014/0107659 A1
(43) Pub. Date: Apr. 17, 2014

(54) IN SITU ROD MEASURING INSTRUMENT AND METHOD OF USE

(71) Applicant: Alphatec Spine, Inc., Carlsbad, CA (US)

(72) Inventors: Carmen Walters, Carlsbad, CA (US); Jens Peter Timm, Carlsbad, CA (US); Kyle Hayes, Mission Viejo, CA (US)

(73) Assignee: Alphatec Spine, Inc., Carlsbad, CA (US)

(21) Appl. No.: 14/043,094

(22) Filed: Oct. 1, 2013

Related U.S. Application Data
(60) Provisional application No. 61/713,045, filed on Oct. 12, 2012.

(51) Int. Cl. A61F 2/46

(52) U.S. Cl. CPC A61F 2/4657
USPC 606/102

(57) ABSTRACT

An instrument for obtaining spinal rod measurements in situ includes a measurement member, a first indicating member, and a second indicating member. The measurement member measures a length between two spinal implants. The first indicating member couples with the measurement member and includes a first measurement scale coupled with a first shaft for engaging a first spinal implant of the two spinal implants. The second indicating member couples with the measurement member and includes a second measurement scale coupled with a second shaft for engaging a second spinal implant of the two spinal implants.

### CROSS-REFERENCE TO RELATED APPLICATIONS

The present application claims priority to U.S. Provisional Application Ser. No. 61/713,045 entitled “Rod Measuring Instruments and Methods of Use” which was filed on Oct. 12, 2012 and is incorporated herein by reference in its entirety.

### FIELD

The invention generally relates to spinal surgery and more particularly to instruments for measuring the required length of stabilization rods.

### BACKGROUND

The spine is a flexible column formed of a plurality of bones called vertebrae. The vertebrae are hollow and piled one upon the other, forming a strong hollow column for support of the cranium and trunk. The hollow core of the spine houses and protects the nerves of the spinal cord. The different vertebrae are connected to one another by means of articular processes and intervertebral, fibrocartilaginous bodies. Various spinal disorders may cause the spine to become misaligned, curved, and/or twisted or result in fractured and/or compressed vertebrae. It is often necessary to surgically correct these spinal disorders.

The spine includes seven cervical (neck) vertebrae, twelve thoracic (chest) vertebrae, five lumbar (lower back) vertebrae, and the fused vertebrae in the sacrum and coccyx that help to form the hip region. While the shapes of individual vertebrae differ among these regions, each is essentially a short hollow shaft containing the bundle of nerves known as the spinal cord. Individual nerves, such as those carrying messages to the arms or legs, enter and exit the spinal cord through gaps between vertebrae.

The spinal disks act as shock absorbers, cushioning the spine, and preventing individual bones from contacting each other. Disks also help to hold the vertebrae together. The weight of the upper body is transferred through the spine to the hips and the legs. The spine is held upright through the work of the back muscles, which are attached to the vertebrae. While the normal spine has no side-to-side curve, it does have a series of front-to-back curves, giving it a gentle “S” shape. If the proper shaping and/or curvature are not present due to scoliosis, neuromuscular disease, cerebral palsy, or other disorder, it may be necessary to straighten or adjust the spine into a proper curvature.

Generally the correct curvature is obtained by manipulating the vertebrae into their proper position and securing that position with a rigid system of screws, rods, intervertebral spaces, and/or plates. The various components of the system may be surgically inserted through open or minimally invasive surgeries. The components may also be inserted through various approaches to the spine including anterior, lateral, and posterior approaches and others in between.

Spinal fixation systems may be used in surgery to align, adjust, and/or fix portions of the spinal column, i.e., vertebrae, in a desired spatial relationship relative to each other. Many spinal fixation systems employ a spinal rod for supporting the spine and for properly positioning components of the spine for various treatment purposes. Vertebral anchors, comprising pins, bolts, screws, and hooks, engage the vertebrae and connect the supporting rod to different vertebrae. The size, length, and shape of the cylindrical rod depend on the size, number, and position of the vertebrae to be held in a desired spatial relationship relative to each other by the apparatus.

During spinal surgery, a surgeon first exposes the spine posterior and attaches the vertebral anchors to selected vertebrae of the spine. The surgeon then inserts a properly shaped spinal rod into rod-receiving portions of the vertebral anchors to connect the selected vertebrae, thereby fixing the relative positions of the vertebrae. Generally, a controlled mechanical force is required to bring together the spinal rod and a spinal implant, such as the vertebral anchors, in a convenient manner. After insertion, a surgeon must insert a locking mechanism, such as a set screw, into the vertebral anchor to lock the spinal rod to the implant after the force for inserting the rod is removed.

Patients suffering from orthopedic injuries, deformities, or degenerative diseases often require surgery to stabilize an internal structure, promote healing, and/or relieve pain. In the spinal field, surgeries to correct spinal abnormalities often involve positioning one or more elongate stabilization elements such as rods, plates or other types of elongate members along a portion of the spinal column, and anchoring each of the elongate stabilization elements to two or more vertebrae via screws, hooks or other types of bone anchors. Prior to anchoring the elongate stabilization element to the spinal column, the surgeon may need to measure the distance between the bone anchors or between two reference locations along the spinal column in order to determine the appropriate length of the elongate stabilization element. In some instances, the bone anchors may be arranged at varying angular orientations, thereby presenting difficulties in accurately measuring the distance between the bone anchors to provide a properly sized elongate stabilization element having a length sufficient for coupling to the bone anchors.

Certain spinal conditions, including a fracture of a vertebra and a herniated disc, indicate treatment by spinal immobilization. Several systems of spinal joint immobilization are known, including surgical fusion and the attachment of pins and bone plates to the affected vertebras. Known systems include screws having proximal heads and threaded shafts that may be inserted into at least two spaced-apart vertebras. Each screw includes a receiver attached over the head such that a stabilization rod can interconnect two or more receivers to immobilize the vertebras spanned by the screws. However, in these systems, a surgeon is unable to visualize the area beneath the skin and determine the proper length at which to cut the stabilization rod prior to insertion. Further, angled placement of screws relative to the surface of the skin introduces additional uncertainty as to the required length of the stabilization rod. Current spinal immobilization systems would therefore benefit from a rod measuring instrument which provides a rapid and accurate measurement of the necessary rod length, utilizing existing surgical sites.

### SUMMARY

A rod measuring instrument according to the principles of the present disclosure includes a first indicating member, a measuring member, and a second indicating member. The first indicating member includes a protractor member and a shaft member. The measuring member includes a first end, a second end, and a length therebetween. The second indicating member also includes a protractor member and a shaft member. The first indicating member is fixedly coupled to the measuring member at the first end and the second indicating member is movably coupled to the measuring member.

In other features, the shaft members of the first and second indicating members are each adapted to removably couple to an installed pedicle screw head. In other features the protractor members of the first and second indicating members indicate a first and second measurement correction value, based on a relative angle of the pedicle screw head to the measuring member. In other features, the protractor members of the first and second indicating members indicate a first and second angle value, based on a relative angle of the pedicle screw head to the measuring member. In still other features the measuring member is marked with linear measurements along its length.

In yet other features, the second indicating member freely slides along the length of the measuring member. In other features, the first and second protractor members each include a body portion and a pointer member. In other features the first and second shaft members includes titanium.

In still other features, the distal tip of the first and second shaft members includes a radiopaque material. In other features, the first and second shaft members further include at least one centering bead disposed along the length of the shaft member, the centering bead adapted to center and stabilize the shaft member within a tissue retractor. In yet other features, the second end of the measuring member is adapted to prevent the second indicating decoupling from the measuring member. In other features, the first and second indicating members further include a double hinge assembly coupled to the shaft member and the protractor member. In still other features, the first and second shaft members further include a height indicator adapted to indicate that the shaft is fully seated in and coupled to the screw head.

In other examples, an instrument for obtaining spinal rod measurements in situ includes a measurement member, a first indicating member, and a second indicating member. The measurement member measures a length between two spinal implants. The first indicating member couples with the measurement member and includes a first measurement scale coupled with a first shaft for engaging a first spinal implant of the two spinal implants. The second indicating member couples with the measurement member and includes a second measurement scale coupled with a second shaft for engaging a second spinal implant of the two spinal implants.

In other features, at least one of the first and second indicating members includes a slidable coupling with the measurement member. In still other features, at least one of the first and second indicating members includes a fixed coupling with the measurement member. In yet other features, at least one of the first and second indicating members includes a pivotal coupling with the measurement member. In still other features, the first shaft and the second shaft include a length greater than a depth of a surgical incision. In yet other features, the first measurement scale indicates an angle of the first shaft relative to the measurement member. In other features, the first measurement scale indicates a length to be added or subtracted from the length indicated by the measurement member.

### BRIEF DESCRIPTION OF THE DRAWINGS

FIG. 1 is an isometric view of an exemplary rod measuring instrument according to the principles of the present disclosure.

FIG. 2 is a side view of the rod measuring instrument illustrated in FIG. 1.

FIG. 3 is a front view of the rod measuring instrument illustrated in FIG. 1.

FIG. 4 is a top view of the rod measuring instrument illustrated in FIG. 1.

FIG. 5 is an isometric view of a shaft of the rod measuring instrument coupled to a pedicle screw.

### DETAILED DESCRIPTION

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein. The words proximal and distal are applied herein to denote specific ends of components of the instrument described herein. A proximal end refers to the end of an instrument nearer to an operator of the instrument when the instrument is being used. A distal end refers to the end of a component further from the operator and extending towards the surgical area of a patient and/or the implant.

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

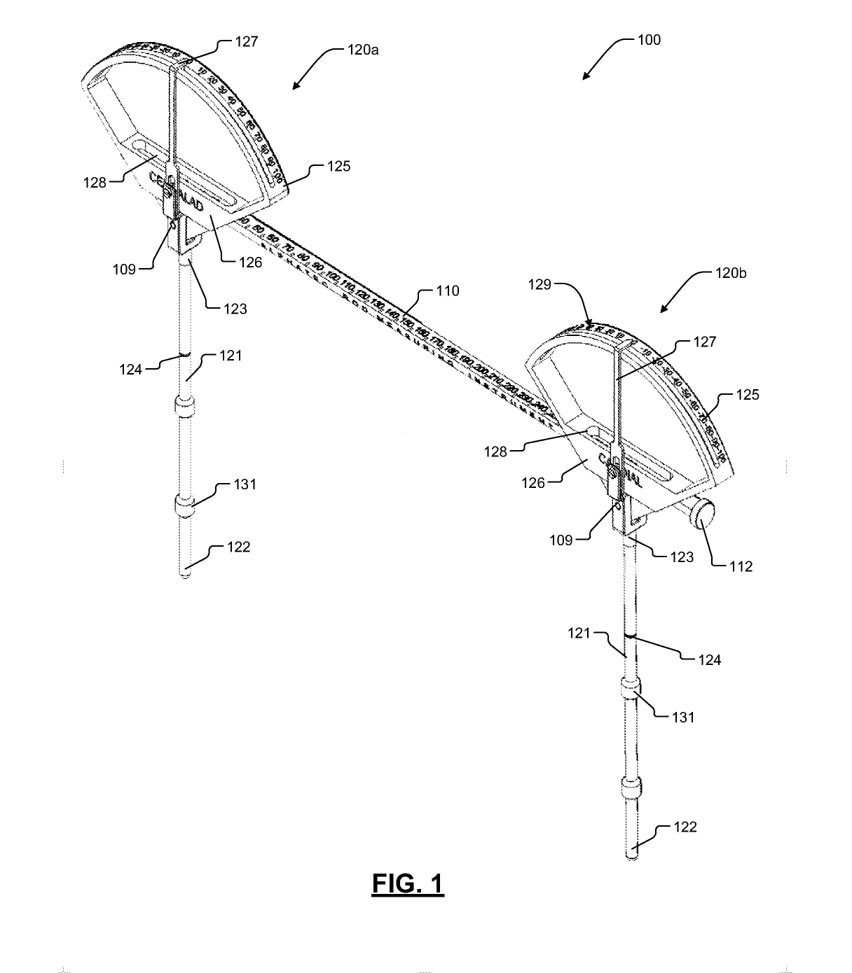

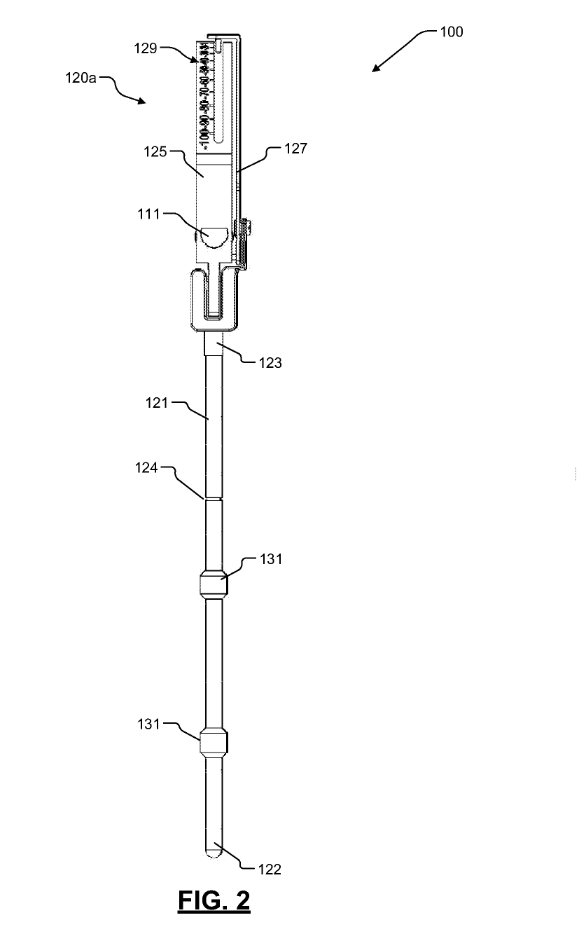

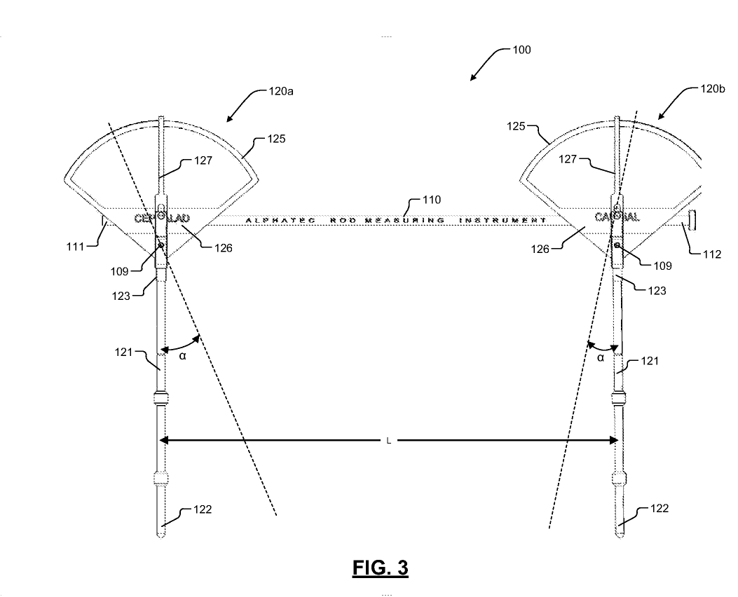

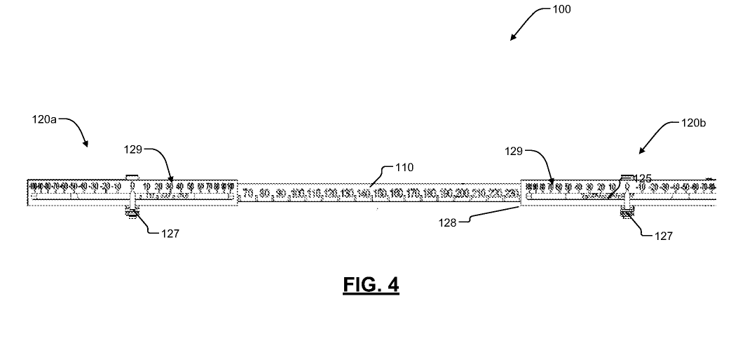

Illustrated in FIGS. 1-5, an exemplary rod measuring instrument 100 includes a first indicating member 120a and a second indicating member 120b. The indicating members (collectively 120) may be substantially identical in features and components. For example, each indicating member 120 may further comprise a protractor member 125 and a shaft member 121. A measuring member 110 including a first end 111, a second end 112, and a length therebetween may link the indicating members 120. The protractor members (collectively 125) each include an aperture, slot, channel, or passageway for sliding engagement with the measuring member 110. The ends 111 and 112 may be adapted to prevent the removal of either or both indicating member 120a and 120b from the measuring member 110.

The measuring member 110 may include any of a variety of cross-sectional profiles including a d-rod, c-channel, cylindrical tube, square rod and the like. The measuring member 110 is marked with linear measurements along its length, such as in a millimeter scale or other alternative measurement scale. In the some examples, the linear measurements are marked on a flat surface of a d-rod shaped measuring member 110. Alternatively, the linear measurements may be marked along any desired portion of a surface of the measuring member 110. The length of the measuring member 110 is selected to permit measurement in a wide variety of surgical treatments, where at least two pedicle screws are utilized. In some embodiments, the measuring member 110 may be interchangeable.

In some examples, the indicating members 120 may both move along the measuring member 110. In other examples, such as in FIG. 1, the first indicating member 120a may be linearly fixed to the measuring member 110 at the first end 111, while maintaining pivotal freedom of the first shaft member 121a relative to the measuring member 110. The linear fixation of indicating member 120a to the measuring member 110 may be achieved by any of a variety of methods, including welding, a set screw, frictional force, glue, locking pins, clips, and/or the like. Non-permanent fixation methods may be advantageous, allowing the instrument 100 to be dismantled for ease of storage when not in use. The second indicating member 120b is movably coupled to the measuring member 110 through the aperture, so as to be capable of linear translation along the length of the measuring member 110. For both indicating members 120, pivotal freedom of the shaft 121 is obtained by a pivotal connection 109, which pivotally couples the shaft 121 to the protractor member 125. For example, the pivotal connection 109 may include a pin connecting the shaft 121 to the protractor member 125.

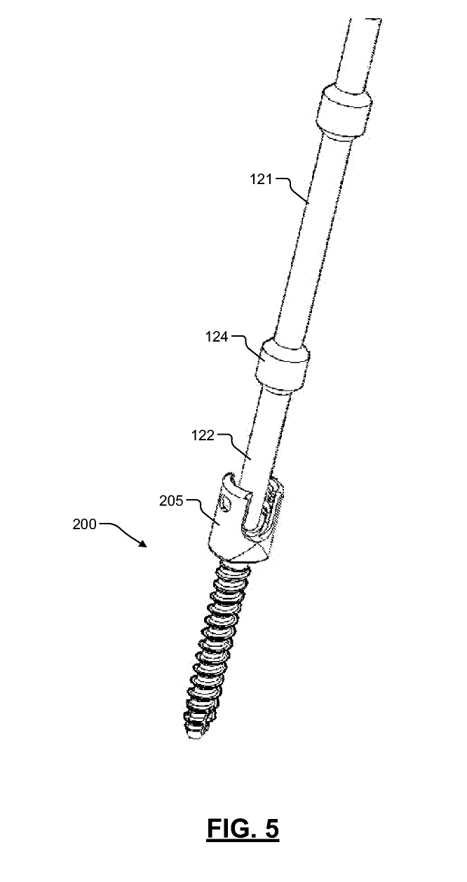

As shown in FIG. 5, the first and second shaft members 121 each have a distal end 122 adapted to be removably coupled to heads 205 of pedicle screws 200 in situ. The shaft members 121 may include a length to permit insertion through a surgical opening in a patient and extend away for ease of use by a surgeon. For example, the length may be substantially greater than a depth of the surgical site measured from the opening on the surface of the skin to the screw 200 within the vertebra. The length may be extendable. For example, the shaft member 121 may comprise a plurality of interchangeable shaft portions coupled together at couplings 124. The couplings 124 may include threaded, snap-fit, or other removable couplings to accommodate patient anatomy and surgeon preference. The first and second shaft members 121 further have a proximal end 123 adapted to be pivotally coupled to the protractor member 125. In another embodiment, the first and second shaft members 121 may have a height indicator (not shown), which lets a user know that the shaft member 121 is properly seated in the head of a pedicle screw. In another embodiment, the first and second shaft members 121 may further comprise one or more center beads 131, which maintain and stabilize the center of the shaft 121 relative to a tissue retractor previously installed in a patient's body

The first and second protractor members 125 each comprise a body portion 126 and a needle 127. The body portion 126 may comprise a viewing port 128, through which the linear position of the protractor member 125 along the measuring member 110 can be determined. This viewing port 128 may be present on both protractor members 125 or on only one protractor member 125. The body portion 126 further comprises a measurement scale 129, which measures the deflection of the needle 127 by the shaft 121 as described herein. The measurement scale 129 may indicate either the angle of deflection a of the shaft 121 or a linear measurement which such angle of deflection represents. When the scale 129 indicates a linear measurement, the scale 129 may further indicate whether the measurement is additive or subtractive from the measurement indicated by the location of the indicating member 120 along the measuring member 110. For example, if a pedicle screw to which the first indicating member 120 is coupled lies beyond the first end 111 of the measuring member 110, then the displacement of the needle 127 along the scale 129 would indicate an additive linear measurement to the location of the first indicating member 120 along the measuring member 110, as measured through the viewing port 128. Alternatively, if the pedicle screw lies within the length of the measuring member 110, then the displacement of the needle 127 along the scale 129 would indicate a subtractive linear measurement.

The needle 127 may be directly coupled to or integral with the shaft 121, so as to cause the needle 127 to displace along the scale 129 as the shaft 121 is angularly deflected. In other examples, the first and second indicating members 125 may utilize a double-hinge assembly to convert small angular movements of the shaft members 121 into larger movements of the needles 127, so that small correction measurements may be accurately read. In the double-hinge embodiments, the needle 127 is pivotally coupled to the body portion 125, at a pivot. The needle 127 is then further pivotally coupled to the proximal end 123 of the shaft 121 by the double hinge assembly. Other known apparatus for amplifying smaller movements of the shafts 121 relative to the measuring member 110 may be used to improve accuracy.

As the shaft members 121 are coupled to the pedicle screws 200 at ends 122 (as shown in FIG. 5), the second indicating member 120 translates along the length of the measuring member 110 as necessary to allow the coupling of the second shaft member 121 and the pedicle screw head 205. The second indicating member 120 is located at a particular linear position along the length of the measuring member 110, such position indicating a particular linear measurement L along the measuring member 110, based on the linear separation of the first and second indicating members 120. The first protractor member 125 serves to convert an angular displacement of the first shaft member 121, and thus the linear displacement of the first pedicle screw, into a first measurement correction value L′. In a preferred embodiment, the first correction value is an additive or subtractive linear value (depending on the direction of angular displacement) in the same scale as the measuring member 110. In an alternative embodiment, the first correction value may only be an angular measurement, and require conversion by a user to a linear value. Similarly, the second protractor member 125 serves to convert the angular displacement of the second shaft member 121, and thus the linear displacement of the second pedicle screw, into a second measurement correction value L″.

Once the linear measurement L, first correction value L′, and second correction value L″ are determined and converted to the same linear scale, the values may be summed to determine the necessary length of the stabilization rod. This length may natively include an arbitrary adjustment to allow for appropriate overhang length of the rod, or such arbitrary adjustment may be included by the user. The stabilization rod of appropriate length may be selected from pre-sized rods, or cut to size from stock rods.

In a further embodiment, the rod measuring instrument 100 may utilize electronic sensors to digitally determine and display the measurements of L, L′, and L″. The electronic elements may utilize any appropriate power source, such as a battery or an electrical outlet. In the above embodiments, the components of the rod measuring instrument 100 may be made of any medically suitable material, as known in the art. The shaft members 121 are preferably made of titanium for optimal imaging. Alternatively, the distal end 122 of the shaft 121 which makes contact with a pedicle screw head may comprise a material suitable for imaging, such as tantalum or gold.

Example embodiments of the methods and systems of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments, but should be defined only in accordance with the following claims and their equivalents.

While the invention has been described in connection with various embodiments, it will be understood that the invention is capable of further modifications. This application is intended to cover any variations, uses, or adaptations of the invention following, in general, the principles of the invention, and including such departures from the present disclosure as, within the known and customary practice within the art to which the invention pertains.

The invention claimed is:

1. A rod measuring instrument, comprising:
a first indicating member, comprising a protractor member and a shaft member;
a measuring member, having a first end, a second end, and a length therebetween; and
a second indicating member, comprising a protractor member and a shaft member,
wherein the first indicating member is fixedly coupled to the measuring member at the first end and the second indicating member is movably coupled to the measuring member.

2. The rod measuring instrument of claim 1, wherein the shaft members of the first and second indicating members are each adapted to removably couple to an installed pedicle screw.

3. The rod measuring instrument of claim 2, wherein the protractor members of the first and second indicating members indicate a first and second measurement correction value, based on a relative angle of the pedicle screw head to the measuring member.

4. The rod measuring instrument of claim 2, wherein the protractor members of the first and second indicating members indicate a first and second angle value, based on a relative angle of the pedicle screw head to the measuring member.

5. The rod measuring instrument of claim 1, wherein measuring member is marked with linear measurements along its length.

6. The rod measuring instrument of claim 1, wherein the second indicating member freely slides along the length of the measuring member.

7. The rod measuring instrument of claim 1, wherein the first and second protractor members each comprise a body portion and a pointer member.

8. The rod measuring instrument of claim 1, wherein the first and second shaft members comprise titanium.

9. The rod measuring instrument of claim 1, wherein a distal dip of the first and second shaft members comprises a radiopaque material.

10. The rod measuring instrument of claim 1, wherein the first and second shaft members further comprise at least one centering bead disposed along the length of the shaft member, the centering bead adapted to center and stabilize the shaft member within a tissue retractor.

11. The rod measuring instrument of claim 1, wherein the second end of the measuring member is adapted to prevent the second indicating decoupling from the measuring member.

12. The rod measuring instrument of claim 1, wherein the first and second indicating members further comprise a double hinge assembly coupled to the shaft member and the protractor member.

13. The rod measuring instrument of claim 2, wherein the first and second shaft members further comprise a height indicator adapted to indicate that the shaft is fully seated in and coupled to the screw head.

14. An instrument for obtaining spinal rod measurements in situ, comprising:
a measurement member for measuring a length between two spinal implants;
a first indicating member coupled with the measurement member and including a first measurement scale coupled with a first shaft for engaging a first spinal implant of the two spinal implants; and
a second indicating member coupled with the measurement member and including a second measurement scale coupled with a second shaft for engaging a second spinal implant of the two spinal implants.

15. The instrument of claim 14, wherein at least one of the first and second indicating members includes a slidable coupling with the measurement member.

16. The instrument of claim 14, wherein at least one of the first and second indicating members includes a fixed coupling with the measurement member.

17. The instrument of claim 14, wherein at least one of the first and second indicating members includes a pivotal coupling with the measurement member.

18. The instrument of claim 14, wherein the first shaft and the second shaft include a length greater than a depth of a surgical incision.

19. The instrument of claim 14, wherein the first measurement scale indicates an angle of the first shaft relative to the measurement member.

20. The instrument of claim 14, wherein the first measurement scale indicates a length to be added or subtracted from the length indicated by the measurement member.
